(19) United States
(12) Patent Application Publication
(10) Pub. No.: US 2022/0280302 A1
(43) Pub. Date: Sep. 8, 2022

(54) ORTHOPAEDIC IMPLANT

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Roger R. Dees, Jr., Drummonds, TN (US); Meghan C. Mendelson, Memphis, TN (US)

(21) Appl. No.: 17/631,249

(22) PCT Filed: Jul. 30, 2020
(86) PCT No.: PCT/US2020/044273

§ 371 Date: Jan. 28, 2022

Related U.S. Application Data
(60) Provisional application No. 62/991,254, filed on Mar. 18, 2020.
(60) Provisional application No. 62/881,544, filed on Aug. 1, 2019.

(51) Int. Cl. A61F 2/30; A61F 2/38

(52) U.S. Cl. CPC A61F 2/30721; A61F 2/3859; A61F 2/389; A61F 2002/30332; A61F 2002/30604

(57) ABSTRACT

A knee prosthesis (10, 20) is disclosed. In one example of an embodiment, the knee prosthesis includes a load bearing component (12, 22) and a stem (14, 24) arranged and configured to be coupled within an intramedullary canal of a patient's bone. In some embodiments, the knee prosthesis may also include a coupler (16) for coupling the stem to the load bearing component. In use, the stem is offset from and angled relative to the load bearing component to facilitate reception of the stem in a bowed or angled intramedullary canal of a patient's tibia or femoral bone. In one example of an embodiment, the coupler may include first (32) and second (34) ends, the central longitudinal axis (33) of the first end and the central longitudinal axis (35) of the second end are offset from one another, non-parallel to one another, and do not intersect or cross each other's path at any point.

### CROSS-REFERENCE TO RELATED APPLICATIONS

This is a non-provisional of, and claims the benefit of the filing date of, pending U.S. provisional patent application No. 62/881,544, filed Aug. 1, 2019, entitled “Orthopaedic Implant,” and is a non-provisional of, and claims the benefit of the filing date of, pending U.S. provisional patent application No. 62/991,254, filed Mar. 18, 2020, entitled “Orthopaedic Implant,” the entirety of each application is incorporated by reference herein.

### FIELD OF THE DISCLOSURE

The present disclosure is directed to an orthopaedic implant and more particularly to a knee prosthesis having an offset and/or angled stem to facilitate placement within an intramedullary canal of a patient's tibia or femur.

### BACKGROUND

Knee arthroplasty or knee replacement procedures generally involve the implantation, installation, etc. (used interchangeably without the intent to limit) of an orthopedic implant such as a knee prosthesis onto a patient's knee. For example, in connection with a total knee replacement, the orthopedic implant (e.g., knee prosthesis) may include a femoral component and a tibial component. In use, the femoral component is attached to the patient's femur while the tibial component is attached to the patient's tibia. Generally speaking, the femoral and tibial components may each include an intramedullary stem, which is attachable to an articular component, a tray, a load bearing component, etc. (terms used interchangeably herein without the intent to limit). In use, the stem is arranged and configured to be inserted within an intramedullary canal of the patient's bone while the tray mounts upon a prepared surface on the patient's bone. A bearing member is typically mounted upon the tray of the tibial component.

Variations in the human anatomy of different patients, especially in bones such as the femur and tibia, creates a need for a variety of implant sizes and configurations. In some instances, for example, the longitudinal axis of a stem of a tibial or femoral component may be laterally offset from the longitudinal axis of the other prosthesis component, such as the tray of the tibial or femoral component. In other words, in order to facilitate improved implantation, the longitudinal axis of the stem is positioned offset with respect to the longitudinal axis of the tray. Moreover, even when offset is provided in the knee implant, there is no uniformity as to the degree or direction of offset as each patient is different.

In addition to a need for offset, some patients require angulation of the stem relative to the tray to account for bowing of the intramedullary canal. For example, across a population of human beings, a valgus bowing of, for example, the tibia exists. Similarly, the intramedullary canal of a patient's femur can bow posteriorly relative to the mechanical axis. Such bowing can limit adequate penetration of the stem into the patient's intramedullary canal resulting in improper positioning of the tibial and femoral components in the knee, and thus may cause pain.

To accommodate bowing, the intramedullary stem of, for example, a femoral component of a knee prosthesis may be angled in the varus/valgus (V/V) direction. Generally speaking, in current prosthesis, the stem is set at a fixed V/V angle coronally. This fixed angle is typically set at about 6 degrees, although some prosthesis utilize a fixed angle of 5 degrees or 7 degrees. In either event, currently, in current prosthesis, the stem is set at a fixed V/V angle. However, due to variations in bone anatomy, the V/V angle can vary from patient to patient. Variations in patient's bone anatomy can also vary sagitally relative to, for example, the articular cartilage geometry (e.g., distal exterior/outside part of the bone) and the position of the intramedullary canal (interior/inside of the bone).

Thus problems arise with optimally positioning, for example, the femoral component for a given patient relative to a mechanical or anatomical axis in regards to V/V and flexion/extension (F/E) while optimally positioning the anterior/posterior (A/P) position, medial/lateral (M/L) position, and to a lesser extent, internal/external (I/E) rotation (on a transverse plain) while rigidly fixing the stem in a position such that the stem fits centrally within the intramedullary canal of the bone.

To address this issue, modular prosthesis systems have been developed in an attempt to accommodate the variability in patient anatomies. Modular systems may include a number of interchangeable parts, each having different sizes or other physical characteristics. Such modular systems are useful in that they allow surgeons to use one or more standard parts with interchangeable components having different characteristics.

For example, U.S. Pat. No. 5,290,313 to Heldreth discloses a modular prosthesis system that includes a prosthetic base portion and a stem extension that is mounted to the undersurface of the base portion. The axis of the main body of the stem (or the elongated stem portion of the stem extension) is offset or spaced from the axis of the mounting portion of the stem extension.

Similarly, U.S. Pat. No. 5,782,920 to Colleran discloses a modular joint prosthesis in which a stem, which is mountable within a patient's bone, is able to be offset from an implant portion through the use of an adapter element. The adapter element includes first and second ends wherein a longitudinal axis extending through the first end is substantially parallel to but offset from a second longitudinal axis extending through the second end of the adapter. The first end of the adapter element has a connection surface that is mateable with a connection surface on a distal end of an elongate extension. The second end of the adapter element is mateable with a proximal end of the elongated stem.

U.S. Pat. No. 6,162,255 to Oyola discloses another joint prosthesis system including parallel and offset devices. Oyola discloses a prosthesis including a tibial component, a collar member, and an elongate stem. The tibial component has an elongate extension member. The collar member is positioned intermediate the extension member and the stem. The prosthesis also includes a bolt member having proximal and distal ends wherein a first longitudinal axis extending through the proximal end of the bolt member is substantially parallel to but offset from a second longitudinal axis extending through the distal end of the bolt member. The proximal end of the bolt member has a bolt head portion that engages the elongate extension member. The distal end of the bolt member extends beyond the distal end of the extension member and attaches to the elongate stem such that the tibial component, collar member and elongate stem are secured to one another. The collar and bolt member may be oriented such that offset is provided in either or both of the medial-lateral and anterior-posterior directions.

The previous devices disclosed in Heldreth, Colleran, and Oyola, however, only address the issue of offset and not angulation. In other words, each of the prior devices only teach axes that are parallel and offset. As noted above, some patients also require angulation of the stem to account for bowing of the intramedullary canal.

U.S. Pat. No. 6,953,479 to Carson et al. attempts to address the issue of angulation by providing an intermediate stem extension that angularly orients an attached tibial or femoral implant stem relative to its corresponding load bearing component (e.g., tibial tray, condylar component, etc.) to facilitate positioning of the stem in a bowed or angled tibial or femoral canal in a manner that allows closer correspondence between the geometry of the implant components and the geometry of the tibia, femur, and knee, and better alignment of the load bearing components with the mechanical axis of the leg.

There remains however a need in the art for a knee prosthesis that allows variable alignment to accommodate for anatomical factors, kinematic alignment variances, or a surgeon-prescribed alignment preference. These factors, variances, or preferences may include items such as human bone deformities, muscle structures, and/or flexion/extension balance, as examples.

It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present invention addresses the issues discussed above by providing a modular prosthesis system. In one embodiment, the modular prosthesis system includes a prosthetic base portion such as, for example, an articular component, a tray, a load bearing component, etc. having a surface for positioning adjacent to a corresponding bone. The base portion including a base mounting member thereon. The modular prosthesis system may further include a stem including a stem extension arranged and configured for insertion into an intramedullary canal or cavity in a patient's bone and a stem mounting member for coupling the stem to the base mounting member. In one embodiment, the stem mounting member has a longitudinal axis and the elongated stem portion has a second longitudinal axis, wherein the second longitudinal axis is nonplanar and non-intersecting (non-divergent) with the longitudinal axis.

In addition, and/or alternatively, in one embodiment, an intermediate stem extension or coupler may be provided for coupling the prosthetic base portion (e.g., a femoral or tibial articular component, tray, articular insert, load bearing component, etc.) with a stem extension. The coupler may be offset from the articular component's connection interface at a known distance, a known orientation, and may be angled relative to the articular component's varus/valgus (V/V) and/or flexion/extension (F/E) desired angular orientation and the desired stem's angular orientation. The invention allows variable alignment to an anatomical or kinematic alignment or to a surgeon-prescribed alignment such as human bone deformities, muscle structures, and/or flexion/extension balance.

In revision knee replacements, the typical femoral component is usually supported by an attached intramedullary (IM) stem. The stem connection mechanism for most revision femoral components are fixed at a set varus/valgus (V/V) angle coronally; typically, about six (6) degrees, although some prosthesis utilize a fixed angle of 5 degrees or 7 degrees. Due to variations in bone anatomy, this varus/valgus (V/V) angle can vary from patient to patient. This variation in bony anatomy also varies sagitally relative to the articular cartilage geometry (i.e. distal exterior/outside part of the bone) and the position of the IM canal (interior/inside of the bone). It is desirable to place the articular part of the femoral component in the most optimal position for a given patient relative to a mechanical or anatomical axis in regards to the V/V angle coronally while optimally positioning the A/P position, M/L position, and to a lesser extent, the internal/external rotation (on a transverse plane) while rigidly fixing a stem to the femoral component in a position such that the stem fits centrally within the intermedullary canal. The intermediate stem coupler of the present invention accomplishes all these goals.

Another embodiment of the invention is a set of intermediate stem couplers having various angulations and/or offsets to meet the specific needs of the surgeon for a particular patient.

Yet another embodiment of the invention is an intermediate stem coupler that is designed on a specific patient basis based on CT, MRI, x-ray and/or other imagery/views of the specific patient receiving the implant.

In one embodiment, a knee prosthesis is disclosed. The knee prosthesis comprises a load bearing component including a first connection mechanism; a stem arranged and configured to be inserted into an intramedullary canal of a patient's bone, the stem including a second connection mechanism; and a coupler including a first end portion having a first central longitudinal axis and a second end portion having a second central longitudinal axis, the first end portion being arranged and configured to engage the first connection mechanism of the load bearing component, the second end portion being arranged and configured to engage the second connection mechanism of the stem so that the coupler couples the stem to the load bearing component; wherein the first central longitudinal axis and the second central longitudinal axis are non-intersecting.

In one embodiment, the first central longitudinal axis and the second central longitudinal axis are non-intersecting, non-parallel, and offset from one another so that the first central longitudinal axis and the second central longitudinal axis are always spaced apart from each other by a distance X.

In one embodiment, the second end portion of the coupler is offset from and angled relative to the first end portion of the coupler to orient the stem relative to the load bearing component to facilitate reception of the stem in a bowed intramedullary canal of the patient's bone.

In one embodiment, the first connection mechanism is an extension portion extending from the load bearing component, the extension portion having an internal bore for receiving the first end portion of the coupler.

In one embodiment, the second end portion of the coupler includes an internal bore for receiving the second connection mechanism of the stem.

In one embodiment, the first connection mechanism of the load bearing component is one of a male taper or a female taper and the first end portion of the coupler includes the other one of a male taper or a female taper for engaging the first connection mechanism; and the second connection mechanism of the stem is one of a male taper or a female taper and the second end portion of the coupler includes the other one of a male taper or a female taper for engaging the second connection mechanism.

In one embodiment, the coupler is integrally formed with one or both of the load bearing component and the stem.

In one embodiment, the coupler is selectively rotationally positioned relative to the load bearing component so that rotation of the coupler adjusts a position of the stem relative to the load bearing component.

In one embodiment, rotation of the coupler adjusts a varus/valgus (V/V) angle, a flexion-extension angle, or a combination thereof, of the stem relative to the load bearing component.

In one embodiment, a kit is disclosed. The kit comprising a plurality of couplers with varying configurations.

In one embodiment, the plurality of couplers include a different offset distance between the first and second central longitudinal axes of the first and second end portions, a different angle between the first and second central longitudinal axes of the first and second end portions, or a combination thereof.

In one embodiment, the coupler is selected from the plurality of couplers via utilization of a lookup table based on measurements of the patient's bone.

In one embodiment, the coupler is selected from the plurality of couplers via a computer assisted surgical system programmed to identify an optimal offset and angle based on the patient's bone.

Embodiments of the present disclosure provide numerous advantages. For example, by providing a coupler having first and second ends with non-intersecting axis, surgeons can movably (e.g., rotational) position the coupler relative to the load bearing component to vary the position of the stem to achieve a desired position of the stem relative to the intramedullary canal of the patient's bone. Additionally, by providing a kit including a plurality of couplers containing varying configurations such as, for example, varying angles, offsets, etc., the surgeon can select a desired coupler from the kit of couplers to further optimize the position of the stem.

Further features and advantages of at least some of the embodiments of the present invention, as well as the structure and operation of various embodiments of the present invention, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 is an exploded, perspective view of an example of an embodiment of a knee prosthesis (e.g., a femoral component or implant) in accordance with one aspect of the present disclosure;

FIG. 2 is a posterior view of the knee prosthesis shown in FIG. 1;

FIG. 3 is an exploded, perspective view of an example of an embodiment of a knee prosthesis (e.g., a tibial component or implant) in accordance with one aspect of the present disclosure;

FIG. 4 is an anterior view of the knee prosthesis shown in FIG. 3;

FIG. 5 is a perspective view of an example of an embodiment of a coupler or an intermediate stem extension that can be used in combination with the knee prosthesis (e.g., femoral implant) of FIG. 1 or the knee prosthesis (e.g., tibial implant) of FIG. 3 in accordance with one aspect of the present disclosure;

FIG. 6 is a cross-section view of the coupler or intermediate stem extension shown in FIG. 5, the cross-sectional view taken along line 6-6 in FIG. 5;

FIG. 7 is a cross-section view of the coupler or intermediate stem extension shown in FIG. 5, the cross-sectional view taken along line 7-7 in FIG. 5;

FIG. 8 is a top view of the knee prosthesis (e.g., femoral component) shown in FIG. 1;

FIG. 9 is a side view of the knee prosthesis (e.g., femoral component) shown in FIG. 1, the femoral component illustrated with the coupler or intermediate stem extension and stem;

FIG. 10 is a posterior view of the knee prosthesis (e.g., femoral component) shown in FIG. 9;

FIG. 11 is a top view of the knee prosthesis (e.g., femoral component) shown in FIG. 9, the femoral component showing a portion of the coupler or intermediate stem extension and stem in cross-section;

FIG. 12 is a side view illustrating multiple example embodiments of the knee prosthesis (e.g., femoral component);

FIG. 13 is a posterior view illustrating multiple example embodiments of the knee prosthesis (e.g., femoral component) shown in FIG. 12;

FIGS. 14A-14F illustrates various views of example embodiments of a stem and a coupler or intermediate stem extension shown with various angle orientations; and

FIGS. 15A-22C illustrate various views of an example embodiment of a coupler on the positioning of a stem relative to a femoral load bearing component in accordance with one or more aspects of the present disclosure. FIGS. 15A-15C illustrate a clock position at noon (or zero), FIGS. 16A-16C illustrate a clock position at 1:30, FIGS. 17A-17C illustrate a clock position at 3:00, FIGS. 18A-18C illustrate a clock position at 4:30, FIGS. 19A-19C illustrate a clock position at 6:00, FIGS. 20A-20C illustrate a clock position at 7:30, FIGS. 21A-21C illustrate a clock position at 9:00, and FIGS. 22A-22C illustrate a clock position at 10:30.

The drawings are not necessarily to scale. The drawings are merely representations, not intended to portray specific parameters of the disclosure. The drawings are intended to depict example embodiments of the disclosure, and therefore are not be considered as limiting in scope. In the drawings, like numbering represents like elements.

### DETAILED DESCRIPTION

Various features, aspects, or the like of an orthopedic implant such as a knee prosthesis will now be described more fully hereinafter with reference to the accompanying drawings, in which one or more aspects of the knee prosthesis will be shown and described. It should be appreciated that the various features, aspects, or the like may be used independently of, or in combination, with each other. It will be appreciated that a knee prosthesis as disclosed herein may be embodied in many different forms and should not be construed as being limited to the embodiments set forth herein. Rather, these embodiments are provided so that this disclosure will convey certain aspects of the knee prosthesis to those skilled in the art. In the drawings, like numbers refer to like elements throughout unless otherwise noted.

As will be described herein, in accordance with one or more aspects of the present disclosure, a knee prosthesis is disclosed. In one embodiment, the knee prosthesis includes an articular component, a tray, a load bearing component, etc. (used interchangeably herein without the intent to limit) and a stem arranged and configured for implantation into an intramedullary canal of a patient's bone such as, for example, the patient's tibia, femur, etc. In one embodiment, the knee prosthesis may also include a coupler positioned between the load bearing component and the stem. In accordance with one or more aspects of the present disclosure, the stem may be offset from and angled relative to the load bearing component to facilitate reception of the stem in a bowed or angled intramedullary canal of a patient's tibia or femur. In one embodiment, this may be accomplished through the interface of the stem and the load bearing component or through the use of an intermediate coupler. In addition, the knee prosthesis can be provided in a set of off-the-shelf designs. Alternatively, the knee prosthesis can be a patient specific design.

As will be described in greater detail herein, in one embodiment, the coupler, which can also be referred to as an intermediate stem extension (terms used interchangeable herein without the intent to limit) couples the stem to the tibial and/or femoral load bearing component of a tibial/femoral orthopedic implant. In one embodiment, the coupler includes a first end portion that engages the tibial and/or femoral load bearing component and a second end portion that engages the stem. In one embodiment, the second end portion of the coupler is offset from and angled relative to the first end portion to orient the stem and thus facilitate reception of the stem in a bowed or angled intramedullary canal of a patient's tibia or femur. In one example of an embodiment, the first end portion of the coupler also has a first central longitudinal axis that does not intersect a second central longitudinal axis of the second end portion.

In one embodiment, the first end portion of the coupler may include, or be in the form of, a male taper that is arranged and configured to engage the tibial and/or femoral load bearing component. The second end portion of the coupler may include, or be in the form of, a female taper that is arranged and configured to engage the stem, although one of ordinary skill in the art would understand that the male and female portions could be switched. Further, other connection mechanisms, such as threads or pinned shaft ends could equally be used. In one embodiment, the coupler can be a set of off-the-shelf designs. Alternatively, the coupler may be a patient specific design (i.e., an implant designed and configured for one particular patient).

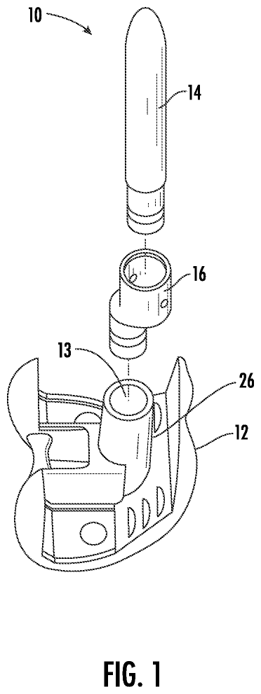

Referring to FIG. 1 an example of an embodiment of a knee prosthesis in accordance with one or more aspects of the present disclosure is shown. As shown, the knee prosthesis may be in the form of a femoral implant 10. The femoral implant 10 may include a femoral load bearing component 12 (e.g., a condylar component), an intermedullary stem 14, and an intermediate stem extension or coupler 16. In one embodiment, the coupler 16 may be omitted and the stem 14 may be directly coupled to the femoral load bearing component 12. In other embodiments, the coupler 16 may be integrally formed with the stem 14 or the femoral load bearing component 12. For example, the stem 14 and the coupler 16 may be monolithic. As illustrated, in use, the coupler 16 connects the femoral load bearing component 12 and the stem 14.

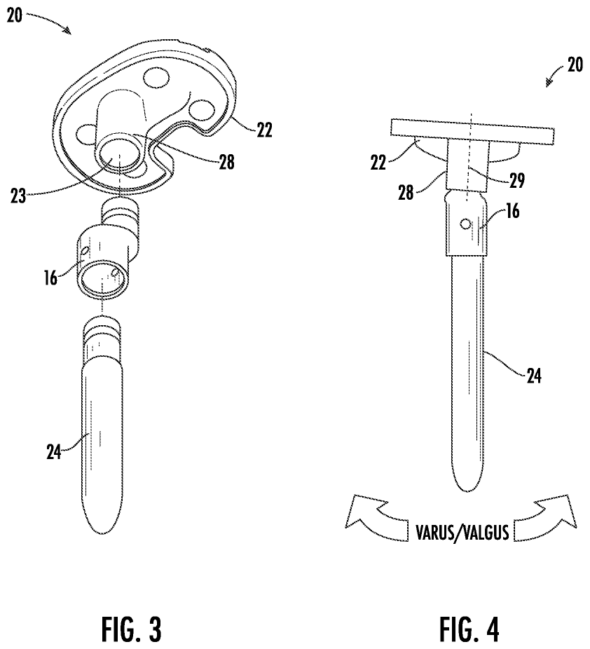

Similarly, referring to FIG. 3 an alternate example of an embodiment of a knee prosthesis in accordance with one or more aspects of the present disclosure is shown. As shown, the knee prosthesis may be in the form of a tibial implant 20. The tibial implant 20 may include a tibial load bearing component 22 (e.g., a tibial tray or platform), an intermedullary stem 24, and an intermediate stem extension or coupler 16. In one embodiment, the coupler 16 may be omitted and the stem 24 may be directly coupled to the tibial load bearing component 22. In other embodiments, the coupler 16 may be integrally formed with the stem 24 or the tibial load bearing component 22. For example, the stem 24 and the coupler 16 may be monolithic. As illustrated, in use, the coupler 16 connects the tibial load bearing component 22 and the stem 24.

As described herein, and as will be appreciated by one of ordinary skill in the art, the stem 14, 24 is arranged and configured to be implanted into an intramedullary canal of a patient's bone such as the patient's femur or tibia. The femoral load bearing component 12 and the tibial load bearing component 22 are arranged and configured to contact an end surface of the patient's femur or tibia, respectively. As described herein, in accordance with one aspect of the present disclosure, the knee prosthesis may include a coupler 16 for coupling the stem 14, 24 to the femoral and tibial load bearing components 12, 24, respectively, the coupler 16 being arranged and configured to provide an offset and/or angled configuration of the stem 14, 24 relative to the femoral and tibial load bearing components 12, 24. While a coupler 16 is shown in the depicted embodiments, those having ordinary skill in the art would understand that the stem 14, 24 and the load bearing components 12, 22 could be constructed and arranged to achieve a monolithic prosthesis that allows offset and/or an angled configuration (e.g., a particular stem angle).

Thereafter, in use, a set of monolithic prosthesis or couplers could be provided so that the desired offset and angle could be selected from within the set of monolithic prosthesis or couplers for the particular patient. Further, those having ordinary skill in the art would understand that the stem 14, 24 and the load bearing component 12, 22 could be constructed and arranged such that the stem 14, 24 is modular and is adapted to connect to the load bearing component at a particular angle. Thereafter, a set of modular stems could be provided so that the desired offset and angle could be selected from within the set for the particular patient.

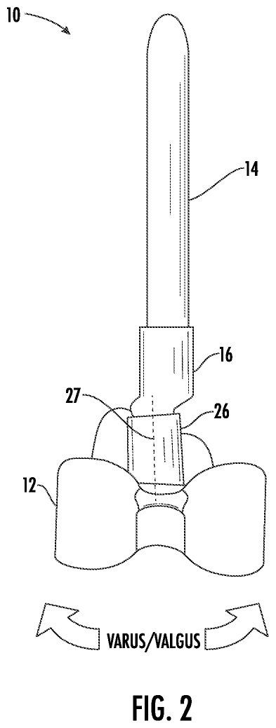

Referring to FIGS. 1 and 2, in the illustrated embodiment, the femoral load bearing component 12 includes an extension portion 26 extending from the femoral load bearing component 12. In use, the extension portion 26 is arranged and configured to couple with the coupler 16. Alternatively, as previously mentioned, the extension portion 26 may be arranged and configured to couple with the stem 14. The extension portion 26 may be coupled to the coupler 16 or stem 14 by any now known or hereafter developed coupling mechanism. For example, as shown, the extension portion 26 may include a cylindrical or frustoconical shape/configuration having a bore 13 arranged and configured to receive at least a portion of the coupler 16 or stem 14, although other shapes/configurations are envisioned. In one embodiment, the bore 13 of the extension portion 26 includes a longitudinal axis 27 (FIG. 2). The longitudinal axis 27 may be arranged and configured at an angle that defines the varus/valgus (V/V) coronal angle used by many surgeons. In the illustrated embodiment, the V/V coronal angle of the extension portion 26 is approximately six (6) degrees, but other angles may be used. While this angle may be different amongst various implant designs, many implant manufacturers use a similar angulation.

Similarly, referring to FIGS. 3 and 4, in the illustrated embodiment, the tibial load bearing component 22 includes an extension portion 28 extending from the tibial load bearing component 22. In use, the extension portion 28 is arranged and configured to couple with the coupler 16. Alternatively, as previously mentioned, the extension portion 28 may be arranged and configured to couple with the stem 24. The extension portion 28 may be coupled to the coupler 16 or stem 24 by any now known or hereafter developed coupling mechanism. For example, as shown, the extension portion 28 may include a cylindrical or frustoconical shape/configuration having a bore 23 arranged and configured to receive at least a portion of the coupler 16 or stem 24, although other shapes/configurations are envisioned. In one embodiment, the bore 23 of the extension portion 28 includes a longitudinal axis 29 (FIG. 4). The longitudinal axis 29 may be arranged and configured at an angle that defines the V/V coronal angle used by many surgeons. In the illustrated embodiment, the V/V coronal angle of the extension portion 28 is approximately six (6) degrees, but other angles may be used such as, for example, five (5) degrees, seven (7) degrees, etc. While this angle may be different amongst various implant designs, many implant manufacturers use a similar angulation. In addition, in the illustrated embodiment, the tibial implant 20 has a posterior slope ranging from zero to ten degrees, and more particularly from three to seven degrees, although other angles may be used. In the embodiment depicted in FIGS. 3 and 4, the tibial implant 20 has a posterior slope of three degrees.

As previously mentioned, some patients require angulation of the stem 14, 24 to account for bowing of the intramedullary canal. As will be described in greater detail below, in accordance with one or more aspects of the present disclosure, the load bearing component 12, 22 and the stem 14, 24 may be arranged and configured to provide an offset and additional angulation to achieve an optimal fit, either in a monolithic or modular form (e.g., incorporating a coupler 16). For example, as shown in the illustrated embodiment and as will be described in greater detail, the coupler 16 may be arranged and configured to provide the offset and desired angulation to provide a better alignment and proper balance of the joint.

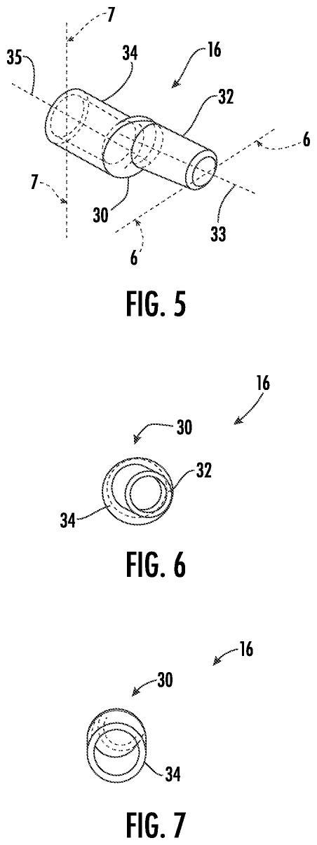

Referring to FIGS. 5-7, an example of an embodiment of a coupler 16 in accordance with one or more aspects of the present disclosure is shown. In one embodiment, the coupler 16 may be made from any suitable material possessing suitable physical properties including structural integrity and adequate strength. In one preferred embodiment, the coupler 16 may be manufactured from an alloy, such as, for example, titanium (Ti-6Al-4V) or cobalt chromium (CoCr). In some embodiments, the coupler 16 may be manufactured via 3D printing or an additive manufacturing technique.

As shown, the coupler 16 may include a body 30 including first and second ends 32, 34. In use, the coupler 16 is arranged and coupled to couple the stem 14, 24 to the femoral and tibial load bearing component 12, 22, respectively. The coupler 16 may include any suitable coupling mechanism for coupling the stem 14, 24 to the femoral and tibial load bearing components 12, 22, respectively, including, for example, threads, adhesive, etc. As shown, in one example embodiment, the first end 32 of the coupler 16 may be in the form of a male taper. The second end 34 of the coupler 16 may be in the form of a female taper, alternatively it is envisioned that the male and female tapers may be reversed. Moreover, in another embodiment, it is envisioned that the first and second ends may both be in the form of male tapers for coupling with female tapers formed on the load bearing component and stem, or vice-versa (e.g., the first and second ends may both include female tapers for coupling with male tapers formed on the load bearing component and stem). In one embodiment, the first end/male taper 32 and the second end/female taper 34 may be integrally formed with the central body 30. Alternatively, it is envisioned that the components may be separately formed and coupled together by any suitable connecting mechanism now known or hereafter developed.

In use, the first end (e.g., male taper) 32 is arranged and configured to couple with the load bearing component 12, 22. For example, as shown in the illustrated embodiments of FIGS. 1-4, the first end (e.g., male taper) 32 may be arranged and configured to be received within the bore 13, 23 formed in the extension portion 26, 28 of the femoral and tibial load bearing components 12, 22, respectively. In use, the second end (e.g., female taper) 34 is arranged and configured to couple with the stem 14, 24, although it is envisioned that the male taper may be arranged and configured to couple to the stem and the female taper may be arranged and configured to couple to the load bearing component. As shown, the second end (e.g., female taper) 34 may be arranged and configured to receive an end portion of the stem 14, 24.

In the illustrated embodiment, the first end (e.g., male taper) 32 of the body 30 has a central longitudinal axis 33. Similarly, the second end (e.g., female taper) 34 of the body 30 has a central longitudinal axis 35. In one example of an embodiment, the central longitudinal axis 33 of the first end (e.g., male taper) 32 and the central longitudinal axis 35 of the second end (e.g., female taper) 34 are offset from one another, non-parallel to one another, and do not intersect or cross each other's path at any point. That is, in accordance with one aspect of the present disclosure, the central longitudinal axes 33, 35 do not intersect (e.g., the central longitudinal axis 33 of the first end (e.g., male taper) 32 is spaced a distance X from the central longitudinal axis 35 of the second end (e.g., female taper) 34 so that they never intersect). Thus arranged, the central longitudinal axis 33 of the first end (e.g., male taper) 32 and the central longitudinal axis 35 of the second end (e.g., female taper) 34 may be referred to as being skewed (e.g., in three-dimensional geometry, the central longitudinal axis 33 of the first end (e.g., male taper) 32 and the central longitudinal axis 35 of the second end (e.g., female taper) 34 do not intersect each other and are non-parallel).

In use, the coupler 16 can be used, for example, when a center of a hole formed in the intramedullary cavity of a patient's bone is not in line with an expected lateral placement of the hole. By using the coupler 16 in accordance with the present disclosure, the angle of the stem 14, 24 relative to the load bearing component 12, 22 can be adjusted, as will be described in greater detail below. Thus, by utilizing a coupler 16 in accordance with the present disclosure, the position, angle, etc. of the stem 14, 24 can be both translationally offset and angularly offset from the load bearing component 12, 22. By appropriately choosing both an angular and a translational offset, the implant stem 12, 24 can be positioned within the reamed hole in the intramedullary canal of the patient's bone without restricting the location and orientation of the articulating surface of the implant (e.g., the load bearing component 12, 22).

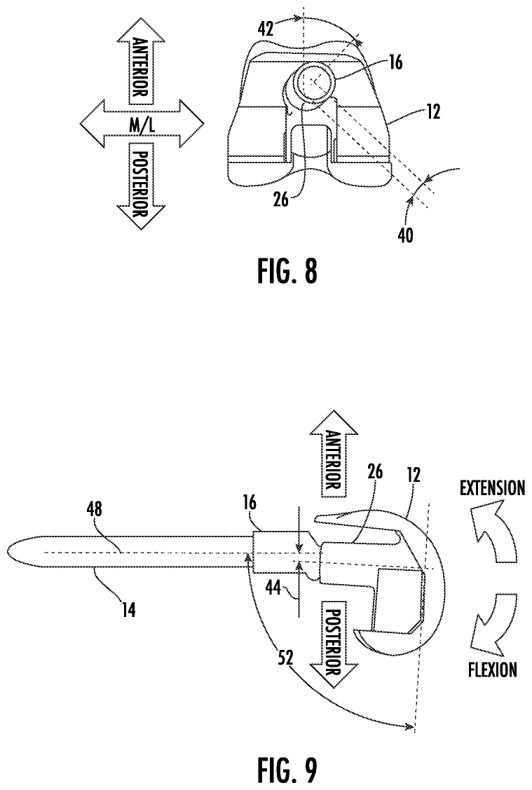

Referring to FIG. 8, in the illustrated embodiment, the coupler 16 includes an offset such as, for example, offset 40 (e.g., space or distance X between the longitudinal axis 33 of the first end 32 and the longitudinal axis 35 of the second end 34). By providing an offset 40, the load bearing component 12, 22 can be moved, shifted, etc., for example, in the medial-lateral position and/or in the anterior-posterior position, relative to the stem 14, 24. In addition, and/or alternatively, referring to FIG. 8, in the illustrated embodiment, each coupler 16 may include a relative angle between the longitudinal axis 33 of the first end 32 and the longitudinal axis 35 of the second end 34 such as, for example, an anterior-posterior angle (e.g., flexion/extension), as schematically shown at 42, relative to the femoral load bearing component 12. By orientating the coupler 16 in the anterior-posterior angle, the load bearing component 12, 22 can be moved, shifted, etc., for example, in the anterior-posterior position relative to the stem 14, 24. In addition, during use, the relative angulation between the longitudinal axis 33 of the first end 32 and the longitudinal axis 35 of the second end 34 enables adjustment of the varus/valgus (V/V) angle/position of the stem 14, 24 relative to the load bearing component 12, 22.

As such, in accordance with one example of an embodiment, a plurality or set of couplers 16 may be provided, for example, in a kit, with couplers 16 having different offsets (40′, 40″, etc.) and different angles (42′, 42″, etc.). As such, as will be described in greater detail below, a user or surgeon can select a desired coupler providing a desired offset and/or angle.

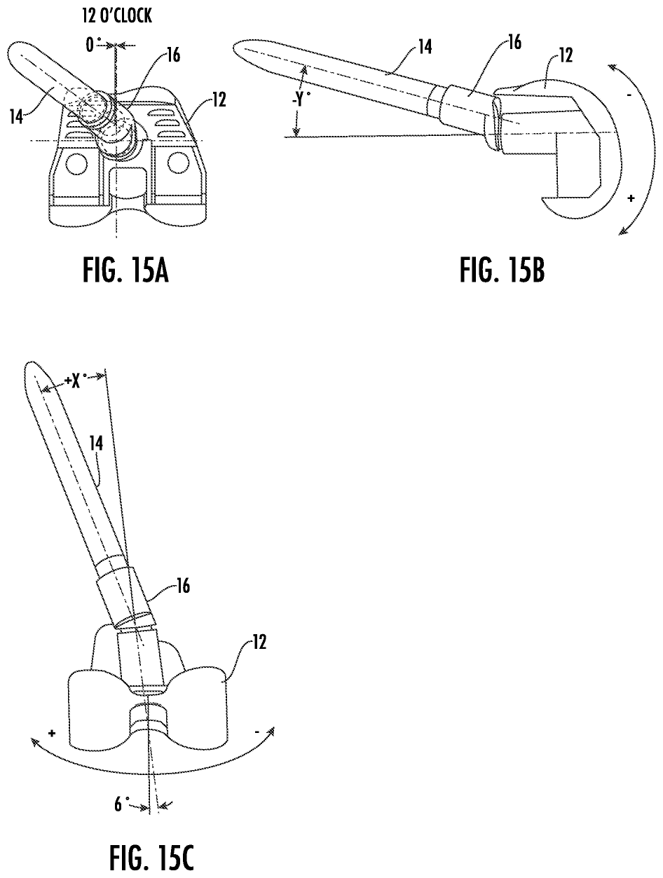

In one embodiment, for each coupler with a desired offset, different couplers having various angles can be provided so that the angle of the stem 14, 24 relative to the load bearing component 12, 22 can be maintained as the coupler 16 is rotated through various clock positions, as will be described in greater detail herein. As such, as will be described in greater detail below, a user or surgeon can select a desired coupler providing a desired offset and angle. In one embodiment, the couplers may be provided with an offset (orientation) of thirty-degrees) (30°) (increments) e.g., kit may be provided with a plurality of couplers equaling 360 degrees, thus, in one embodiment, if a kit is provided with couplers containing an offset of thirty-degree increments, twelve couplers having various angles may be provided. In this manner, as the coupler with thirty-degree offset is rotated amongst the various clock positions, the desired coupler providing the desired angle at the selected clock position can be selected. Alternatively, it is envisioned that the couplers may be provided with different offsets such as, for example, forty-five degrees (45°) increments, sixty-degrees (60°) increments, ninety-degrees (90°) increments, fifteen-degrees (15°) increments, or the like. For example, the couplers may be provided in a kit with increments in the orientation angle. Thus, a kit with the same V/V angle (represented by angle X in the FIGS. 15A-22C) and/or F/E angle (represented by angle Y in the FIGS. 15A-22C) at each clocking orientation increment. This allows the surgeon to select V/V and/or F/E correction amount independent of the offset direction/orientation to provide the desired M/L and/or A/P adjustment.

Generally speaking, as will be appreciated by one of ordinary skill in the arts, by manufacturing and offering smaller increments, a larger number of components is required. Thus, a balancing act is required. In one example of an embodiment, 2 mm, 4 mm, and 6 mm increments can be provided. For these increments, a range of V/V angle adjustments and F/E angle adjustments can be provided. In one embodiment, an initial range is determined. Then an increment of angle for each can be selected followed by the number of clocking orientations. Thus, by way of a non-limiting example, 4 degrees of V/V angle (X°) and 4 degrees of F/E angle (Y°) and an incremental coverage of 2 degrees and a clocking orientation of 45 degrees could be provided in a kit including: V/V angle (X°)=2 degrees at 8 orientations (e.g., clocking orientations 12:00, 1:30, 3:00, 4:30, 6:00, 7:30, 9:00, and 10:30); V/V angle (X°)=4 degrees at 8 orientations; F/E angle (Y°)=2 degrees at 8 orientations; F/E angle (Y°)=4 degrees at 8 orientations; V/V angle (X°) and F/E angle (Y°)=2 degrees at 8 orientations; V/V angle (X°) and F/E angle (Y°)=4 degrees at 8 orientations; V/V angle (X°)=2 degrees and F/E angle) (Y°)=4 degrees at 8 orientations; and V/V angle (X°)=4 degrees and F/E angle (Y°)=4 degrees at 8 orientations. For opposite and negative angles, there would be orientated 180 degrees.

For example, referring to FIGS. 15A-22C, the effects on the positioning of the stem 14 relative to the femoral load bearing component 12 are illustrated. Although not illustrated, the effects on the positioning of the stem 24 relative to the tibial load bearing component 22 would be similar. In the example embodiment, a coupler 16 having an offset (orientation) of forty-five degrees (45°) is illustrated. As illustrated, by rotating the coupler 16 through various positions (e.g., similar to positions on a clock, thus referred to herein as clock-positions), the position of the stem 14 can be adjusted relative to the femoral load bearing component 12. In addition, as illustrated, the varus/valgus (V/V) angle of the stem 14 relative to the femoral load bearing component 12 and the anterior/posterior (A/P) angle of the stem 14 relative to the femoral load bearing component 12 is adjusted.

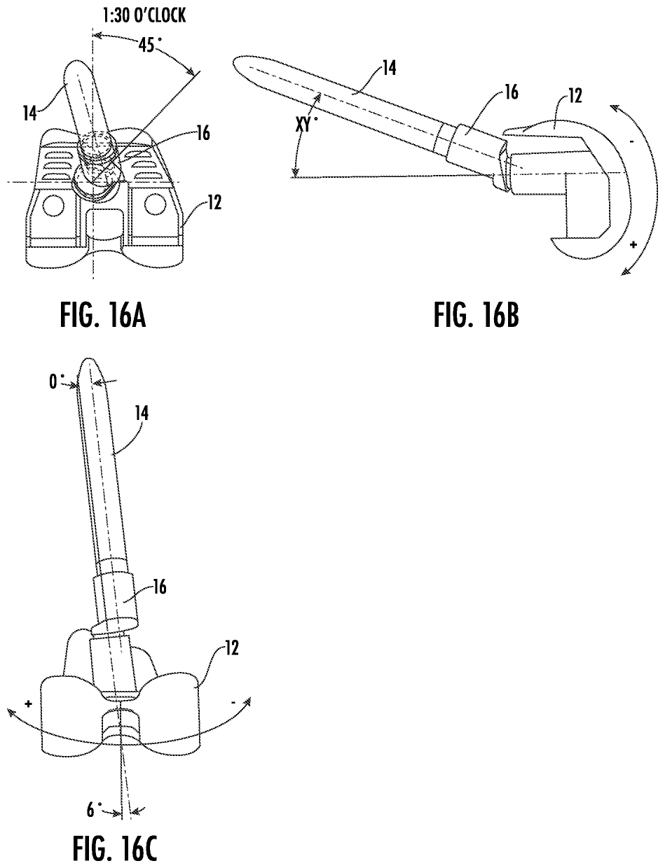

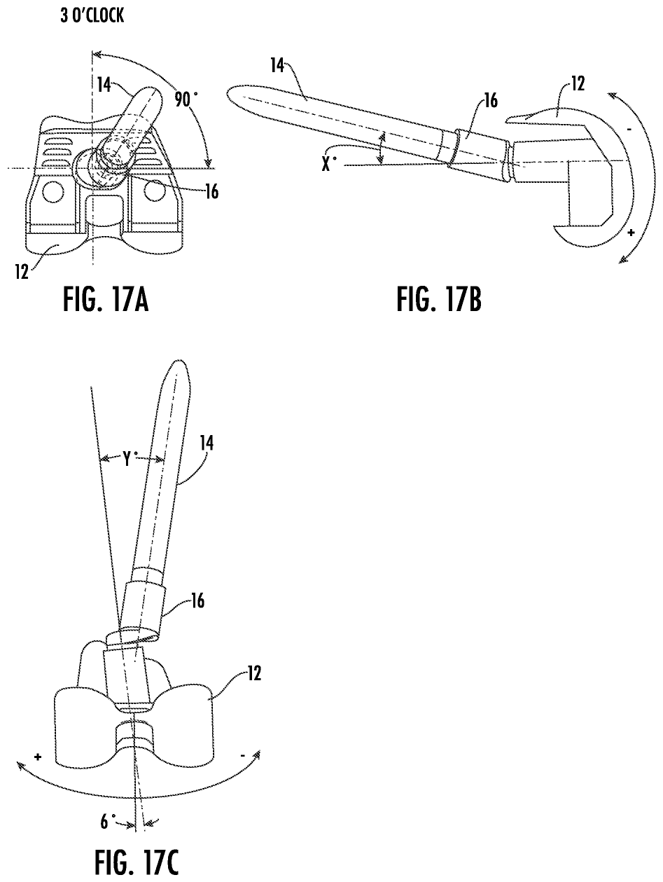

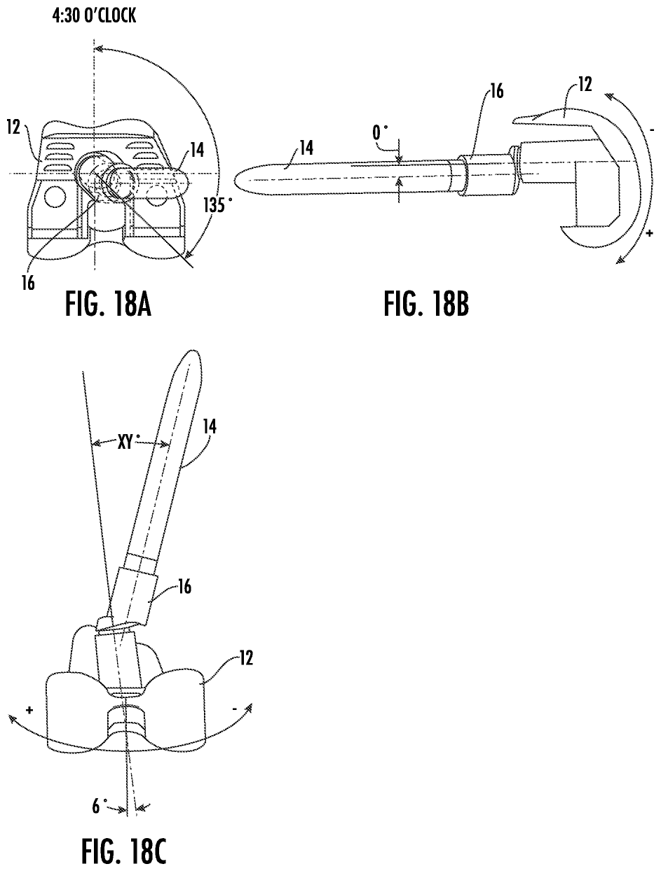

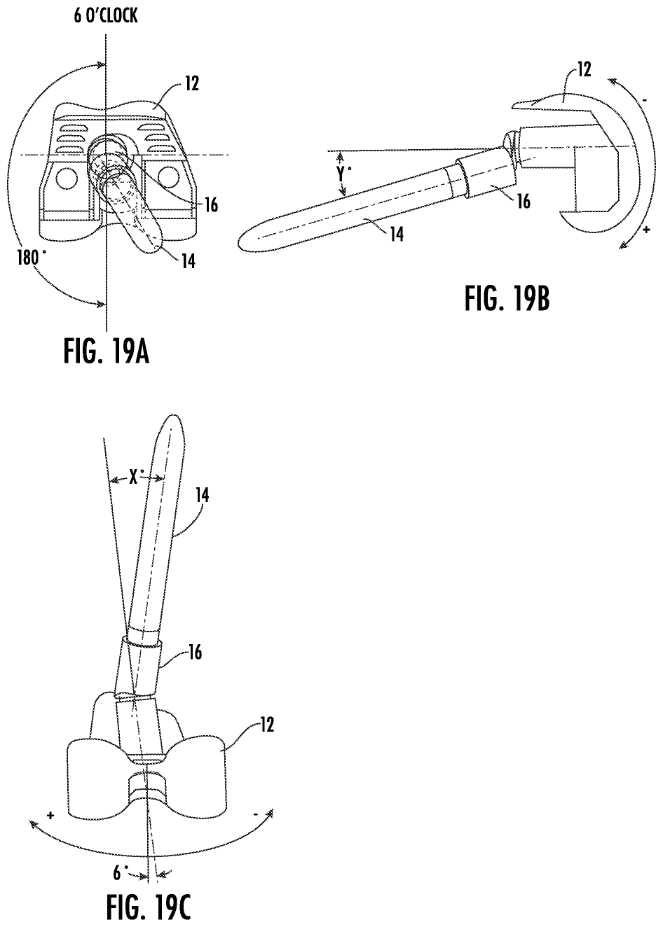

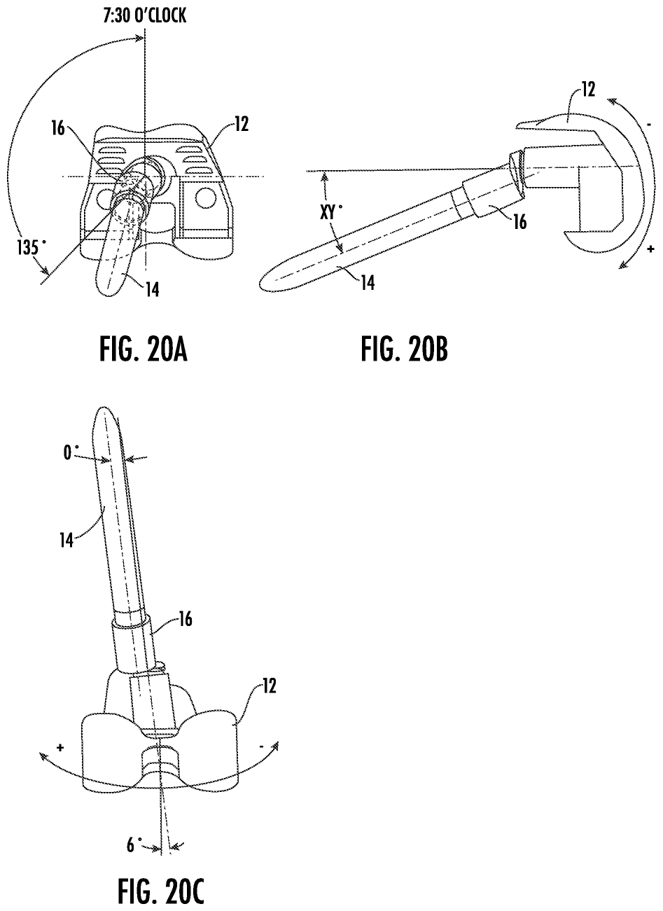

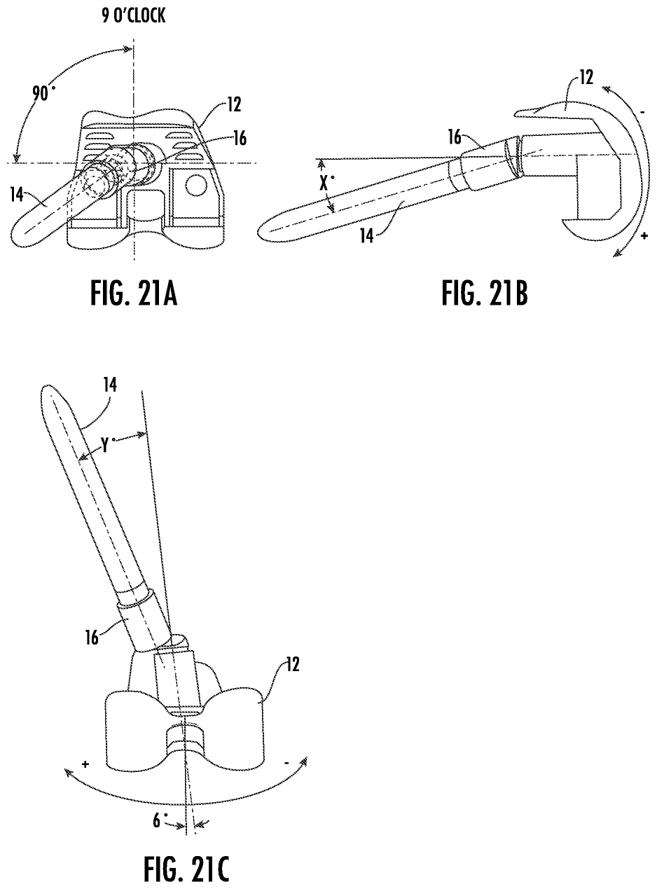

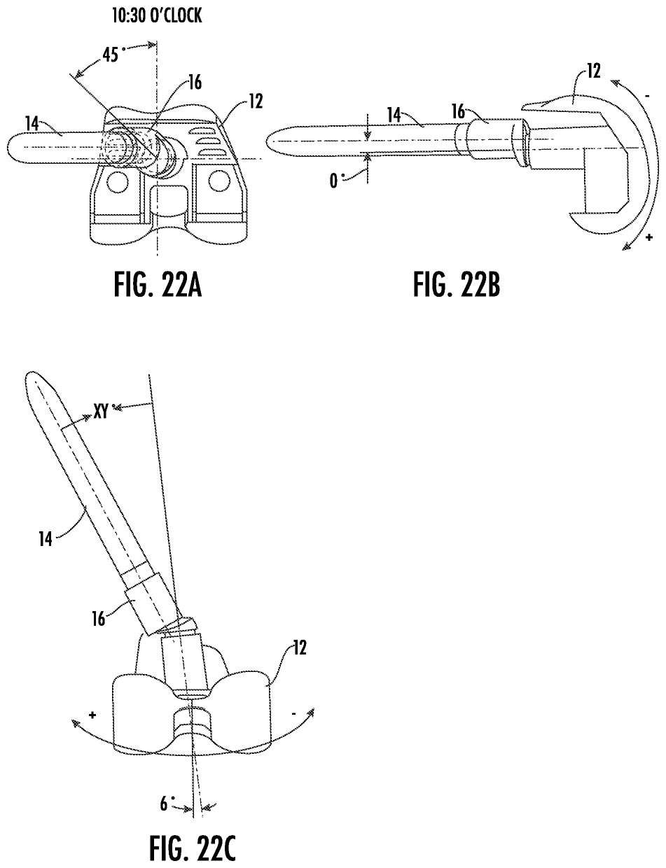

FIGS. 15A-15C illustrate a clock position at noon (or zero). FIGS. 16A-16C illustrate a clock position at 1:30. FIGS. 17A-17C illustrate a clock position at 3:00. FIGS. 18A-18C illustrate a clock position at 4:30. FIGS. 19A-19C illustrate a clock position at 6:00. FIGS. 20A-20C illustrate a clock position at 7:30. FIGS. 21A-21C illustrate a clock position at 9:00. FIGS. 22A-22C illustrate a clock position at 10:30. As illustrated, by utilizing a coupler 16 with an offset, rotation of the coupler 16 along the various clock-positions alters the varus/valgus (V/V) angle of the stem 14 relative to the femoral load bearing component 12 and/or the flexion/extension (F/E) angle of the stem 14 relative to the femoral load bearing component 12. By selecting the appropriate coupler 16 having the desired angulation for the particular clock position, the medial/lateral (M/L) position and the anterior-posterior position of the stem 14 relative to the femoral load bearing component 12. It should be noted, in use, at certain orientations such as, for example, 180 degrees apart, the stem may result in a parallel orientation to V/V (corona) plane or F/E (sagittal) plane thereby not affecting the V/V or F/E load bearing component angle at that orientation. See, for example, FIGS. 16C and 20C, clocking positions 1:30 and 7:30, have no change to V/V, but the F/E is affected. FIGS. 18B and 22B, clocking positions 4:30 and 10:30 have no change to F/E, but the V/V is affected.

As will be appreciated, the kit may include a plurality of couplers 16 having different offsets 40 and different angles 42. Moreover, the kit may include a plurality of couplers 16 with each having a different offset (40′, 40″, etc.) and also each having a different angle (42′, 42″, etc.), wherein, a surgeon may select the coupler 16 with the desired combined offset 40 and angle 42 from the set. This allows the surgeon to select an appropriate offset for the coupler from zero to some distance that will cover all reasonable situations.

In one embodiment, the kit may be provided with any number, configuration, etc. of couplers. For example, in one embodiment, the kit includes a plurality of couplers wherein at least one of the couplers includes a central longitudinal axis 33 of the first end (e.g., male taper) 32 and a central longitudinal axis 35 of the second end (e.g., female taper) 34 that are offset from one another, non-parallel to one another, and do not intersect or cross each other's path at any point, as previously described. In one embodiment, the kit may also include one or more additional traditional couplers that, for example, include parallel axes, or the like.

In accordance with one aspect of the present disclosure, the extension portion 26, 28 is angled relative to the load bearing component 12, 22 by, for example, a fixed angle such as, for example, six (6) degrees. In addition, the first and second ends 32, 34 of the coupler 16 are angled relative to each other and/or offset. Thus arranged, in use, by selecting a desired coupler 16 containing, for example, the desired angle and/or offset, and by rotating the coupler 16 relative to the extension portion 26, 28, rotation of the coupler 16 alters the angle of the stem 14, 24 relative to the load bearing component 12, 22 in, for example, the V/V direction and the A/P direction. As such, the surgeon can selectively position the stem 14, 24 to best fit the intramedullary canal of each patient.

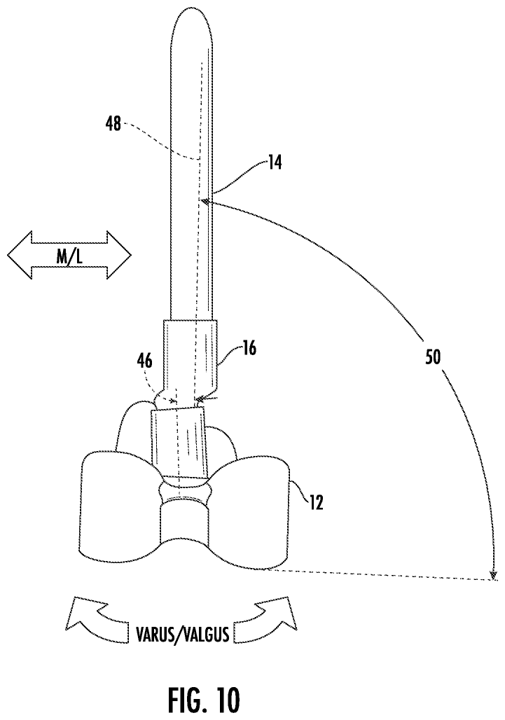

Referring to FIGS. 9 and 10, in accordance with one aspect of the present disclosure, a coordinate system may be established to define a central longitudinal axis 48 of the stem 14. In use, with the coupler 16 coupled to the extension portion 26 extending from the femoral load bearing component 12, an anterior-posterior angle 44 of the stem 14 relative to the load bearing component 12 and a V/V angle 46 of the stem 14 relative to the load bearing component 12 can be defined. By selecting a desired coupler 16 including a desired offset 40 and/or a desired angle, the surgeon can achieve the desired anterior-posterior and medial-lateral position of the stem 14 relative to the femoral load bearing component 12 to provide a desired stem-to-intramedullary canal engagement axis for the stem 14 relative to the intramedullary canal of the patient's bone.

Thus arranged, the coupler 16 is positioned at an angle (e.g., a compound angle) (Ya, Xa) relative to the desired stem-to-intramedullary canal engagement axis 48 defining a varus/valgus 50 (FIG. 10) angle between the femoral component 12 and the engagement axis 48 and a flexion/extension 52 (FIG. 9) angle between the femoral component 12 and the engagement axis 48. In contrast with current, known knee prosthesis that maintain the angles 50, 52 at a fixed dimension (e.g., generally speaking, most current systems maintain the varus/valgus angle 50 at six (6) degrees), the coupler 16 in accordance with one or more principles of the present disclosure enable the surgeon to vary the angles 50, 52 to optimize the position of the stem relative to the patient's intramedullary canal by rotating the position of the coupler 16 relative to, for example, the extension portion 26.

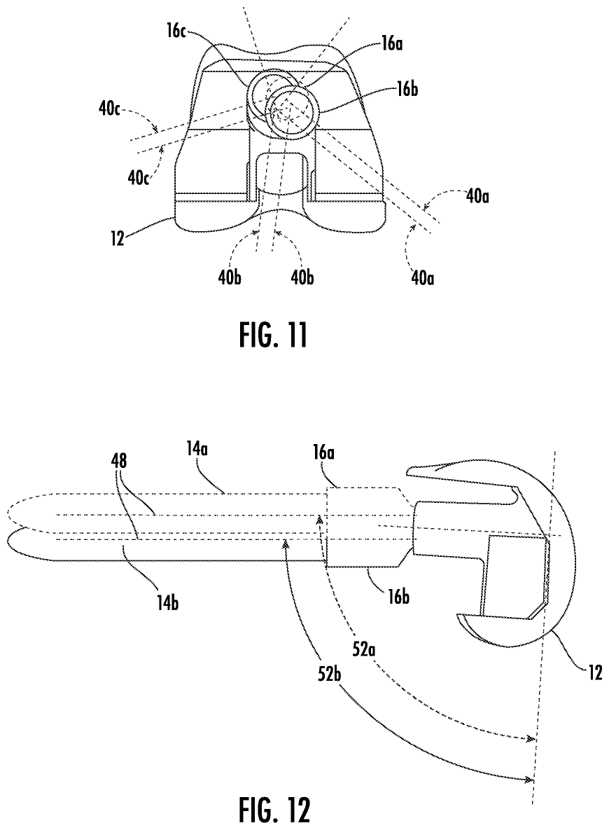

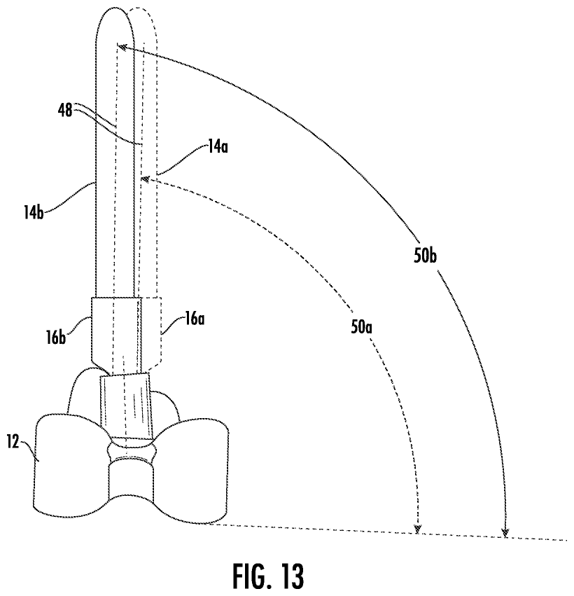

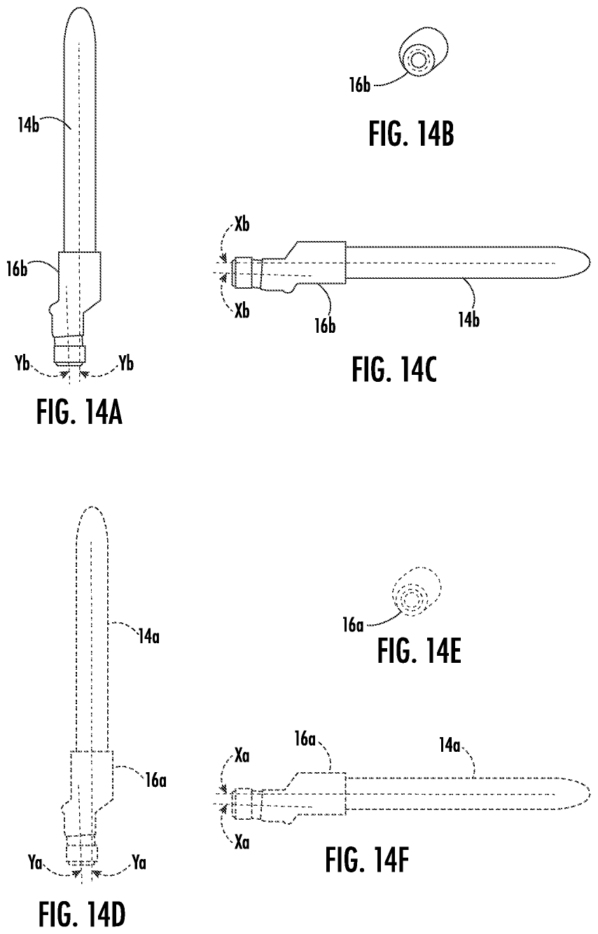

Referring to FIGS. 9-14, in the illustrated embodiment, the coupler 16 can have a first varus/valgus angle 50 and/or a flexion/extension angle 52 when utilizing a coupler having a first offset 40a and a first offset angle 42a orientation. Thereafter, the offset angle (42b, 42c, etc.) can be adjusted via, for example, rotation of the coupler 16, which changes the orientation of the coupler 16 relative to the load bearing component causing the angles for varus/valgus (Xb, Yb) and/or flexion/extension (Xb, Yb) to change to reproduce the same first varus/valgus (50a) angle and/or flexion/extension (52a) angle/compound angle at the first offset angle orientation (42a). In some embodiments, as previously mentioned, a plurality of couplers 16 having varying characteristics can be provided, a coupler repeating the same first varus/valgus (50b, etc. parallel to 50a) and/or flexion/extension (52b, etc. parallel to 52a) angle/compound angle with only a differing amount of offset (40). This plurality of couplers provides a first set of couplers defined with the first varus/valgus (50) and/or flexion/extension (52) angle/compound angle. Additionally, a plurality of sets of various sets of couplers defined by the same as described with a second and third and so on set varus/valgus (50, differing value of 50a) and/or flexion/extension (52, differing value of 52a) angles/compound angles may be provided.

In one example of an embodiment, as previously mentioned, a coupler or a plurality of couplers can be provided with predetermined characteristics such as, for example, offsets, angles, etc. Alternatively, in one example of an embodiment, a patient specific coupler can be achieved. For example, patient specific information could be obtained through imaging, such as by an MRI. Thereafter, by determining a desired offset, offset orientation, varus/valgus orientation, flexion/extension orientation, etc., the patient specific coupler can be manufactured via, for example, 3D printing.

In one example of an embodiment, identification of the desired offset and angle may be achieved via a mechanical apparatus. For example, analog measurements may be taken relative to the intramedullary canal. For example, in one embodiment, a load bearing component such as, for example, the tibial load bearing component or the femoral load bearing component, may be tentatively positioned in its desired location. Thereafter, the surgeon may measure the angles of the load bearing component relative to the A/P and/or M/L position and the V/V and/or F/E position of the intramedullary canal. Alternatively, in one example of an embodiment, a computer assisted surgical system may be used to identify the optimal offset and angle for a particular patient. For example, in one example of a method, a plurality of couplers may be provided in a kit. Thereafter, based on the obtained patient-specific measurements, whether taken mechanically by the surgeon or by a computer assisted surgical system, the data/information can be used to select the optimum coupler from the plurality of provided couplers for the specific patient. In one embodiment, the data/information can be uploaded into a computer system for determining the optimum coupler. In an alternate embodiment, a table or lookup chart can be provided for assisting the surgeon in selecting the optimum coupler. Thus arranged, based on data/information obtained for the specific patient, a preferred varus/valgus angle between the load bearing components 12, 22 and the engagement axis of the stem 14, 24 and a preferred flexion/extension angle between the load bearing components 12, 22 and the engagement axis of the stem 14, 24 can be determined. Utilizing either a computer system or a lookup table, the appropriate coupler for achieving the angles can be selected and achieved. An exemplary computer assisted surgical system for assisting with identifying the proper coupler having an angular and offset between the articulating surface of the load bearing components and the stem is described in U.S. Provisional Patent Application No. 62/961,304, filed on Jan. 15, 2020, entitled “Trial-First Measuring Device for Use During Revision Total Knee Arthroplasty,” which is incorporated herein by reference in its entirety. In use, measurements needed to select the desired coupler will be dependent on the technique, planning and intra-op assessment method being used. For example, computer assisted, robotic, mechanical instruments, forms of pre-op planning (e.g. patient specific instruments), and/or desired corrections prescribed by the surgeon. In one embodiment, from the desired articular implant orientation, measurements include stem offset and orientation, V/V angle and F/E angle relative to the articular implants fixed stem axis and location.

Terms such as proximal, distal, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments. Rather these embodiments should be considered as illustrative and not restrictive in character. All changes and modifications that come within the spirit of the invention are to be considered within the scope of the disclosure. The present disclosure should be given the full scope defined by the language of the following claims, and equivalents thereof.

The foregoing description has broad application. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with another embodiment to yield a still further embodiment. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. Moreover, the following claims are hereby incorporated into this Detailed Description by this reference, with each claim standing on its own as a separate embodiment of the present disclosure.

1. A knee prosthesis comprising:
a load bearing component including a first connection mechanism;
a stem arranged and configured to be inserted into an intramedullary canal of a patient's bone, the stem including a second connection mechanism; and
a coupler including a first end portion having a first central longitudinal axis and a second end portion having a second central longitudinal axis, the first end portion being arranged and configured to engage the first connection mechanism of the load bearing component, the second end portion being arranged and configured to engage the second connection mechanism of the stem so that the coupler couples the stem to the load bearing component;
wherein the first central longitudinal axis and the second central longitudinal axis are non-intersecting.

2. The knee prosthesis of claim 1, wherein the first central longitudinal axis and the second central longitudinal axis are non-intersecting, non-parallel, and offset from one another so that the first central longitudinal axis and the second central longitudinal axis are always spaced apart from each other by a distance X.

3. The knee prosthesis of claim 1, wherein the second end portion of the coupler is offset from and angled relative to the first end portion of the coupler to orient the stem relative to the load bearing component to facilitate reception of the stem in a bowed intramedullary canal of the patient's bone.

4. The knee prosthesis of claim 1, wherein the first connection mechanism is an extension portion extending from the load bearing component, the extension portion having an internal bore for receiving the first end portion of the coupler.

5. The knee prosthesis of claim 4, wherein the second end portion of the coupler includes an internal bore for receiving the second connection mechanism of the stem.

6. The knee prosthesis of claim 1, wherein:
the first connection mechanism of the load bearing component is one of a male taper or a female taper and the first end portion of the coupler includes the other one of a male taper or a female taper for engaging the first connection mechanism; and
the second connection mechanism of the stem is one of a male taper or a female taper and the second end portion of the coupler includes the other one of a male taper or a female taper for engaging the second connection mechanism.

7. The knee prosthesis of claim 1, wherein the coupler is integrally formed with one or both of the load bearing component and the stem.

8. The knee prosthesis of claim 1, wherein the coupler is selectively rotationally positioned relative to the load bearing component so that rotation of the coupler adjusts a position of the stem relative to the load bearing component.

9. The knee prosthesis of claim 8, wherein rotation of the coupler adjusts a varus/valgus (V/V) angle, a flexion-extension angle, or a combination thereof, of the stem relative to the load bearing component.

10. A kit according to claim 1, wherein the kit comprises a plurality of couplers with varying configurations.

11. The kit of claim 10, wherein the plurality of couplers each include a different offset between the first and second central longitudinal axes of the first and second end portions, a different angle between the first and second central longitudinal axes of the first and second end portions, or a combination thereof.

12. The kit of claim 11, wherein the coupler is selected from the plurality of couplers via utilization of a lookup table based on measurements of the patient's bone.

13. The kit of claim 11, wherein the coupler is selected from the plurality of couplers via a computer assisted surgical system programmed to identify an optimal offset and angle based on the patient's bone.
